(19) United States
(12) Patent Application Publication
(10) Pub. No.: US 2023/0104018 A1
(43) Pub. Date: Apr. 6, 2023

(54) CARDIOVASCULAR DISEASE RISK ANALYSIS SYSTEM AND METHOD CONSIDERING SLEEP APNEA FACTORS

(71) Applicant: NDUSTRY-ACADEMIC COOPERATION FOUNDATION, YONSEI UNIVERSITY, Seoul (KR)

(72) Inventors: Joon Sang LEE, Seoul (KR); Young Woo KIM, Seoul (KR); Jun Hong KIM, Seoul (KR)

(21) Appl. No.: 17/532,167

(22) Filed: Nov. 22, 2021

(30) Foreign Application Priority Data
Oct. 5, 2021 (KR) 10-2021-0131598

(51) Int. Cl. A61B 5/00; A61B 6/00; A61B 5/021; A61B 5/026; A61B 5/02; G16H 50/30; G16H 50/50

(52) U.S. Cl. CPC A61B 5/7275; A61B 5/4818; A61B 6/50; A61B 6/5217; A61B 5/021; A61B 5/026; A61B 5/02035; A61B 5/7278; G16H 50/30; G16H 50/50

(57) ABSTRACT

Provided is a cardiovascular disease risk analysis system and method considering sleep apnea factors to analyze a cardiovascular disease risk of sleep apnea patients.

### CROSS-REFERENCE TO RELATED APPLICATIONS

This application claims priority under 35 U.S.C. § 119 to Korean Patent Application No. 10-2021-0131598, filed on Oct. 5, 2021, in the Korean Intellectual Property Office, the disclosure of which is incorporated herein by reference in its entirety.

### TECHNICAL FIELD

The following disclosure relates to a cardiovascular disease risk analysis system and method considering sleep apnea factors, and in particular, to a cardiovascular disease risk analysis system and method considering sleep apnea factors to calculate a patient's sleep apnea severity diagnostic variable value using the patient's biometric information and analyzing cardiovascular disease risk of the corresponding patient using the patient's sleep apnea severity diagnostic variable value.

### BACKGROUND

Cancer, heart disease, and pneumonia, which are the three leading causes of death in Korea as of 2019, account for 45.9% of the total deaths, and among them, heart disease is the second highest cause of death. On a global scale, heart disease is the number one cause of death.

Medical expenses due to domestic circulatory system diseases have been increased at an average annual rate of 8.4% since 2015, reaching about 10.5 trillion won in 2019. The global stent market is on the order of about $7.98 billion as of 2016 and is forecast to grow at an average annual rate of 3.8% for the next five years. As such, interest in heart disease has increased and a size of a related market has also increased significantly.

When cardiovascular disease occurs, myocardial ischemia may be alleviated through a vasodilation procedure based on a stent, and a doctor decides whether to perform the stent procedure generally by calculating a fractal flow reserve (FFR) of a target patient, specifically, a pressure ratio of a lesion-based proximal and distal on the basis of 0.8. Various methods may be used to measure such FFR, and there are mainly an invasive measurement method through a catheter and a non-invasive measurement method that performs vascular modeling through a computational fluid.

The invasive measurement method through a catheter, which measures pressure by directly inserting a pressure sensor into a blood vessel, is known as the most accurate method of FFR measurement, and a decision on stent insertion is made in a final clinical stage. This method is disadvantageous in that it incurs high measurement cost and a location of the lesion should be accurately known in advance.

In the case of the non-invasive measurement method that performs vascular modeling through computational fluid, after CT image modeling of a target patient is performed, a computational fluid analysis is performed. In this method, various flow analyses (WSS, OSI, blood pressure, blood flow, etc.), as well as FFR, may be performed, which has the advantage of locating a lesion through analysis. This method, however, requires a computational fluid analysis time and skilled professionals for analysis.

### RELATED ART DOCUMENT

### Patent Document

Korean Patent Registration No. 10-2003412 (Registration date: Jul. 18, 2019)

### SUMMARY

An exemplary embodiment of the present disclosure is directed to providing a cardiovascular disease risk analysis system and method considering sleep apnea factors, capable of performing a cardiovascular disease risk analysis for overall cardiovascular pre-screening and treatment planning by calculating a patient's sleep apnea severity diagnostic variable value using the patient's biometric information that is easy to collect and performing computational fluid analysis simulation through setting of boundary conditions for vessel/blood flow modeling using the calculated sleep apnea severity diagnostic variable value.

In one general aspect, a system for analyzing a cardiovascular disease risk of a sleep apnea patient includes: a biometric information input unit receiving biometric information of a target patient; a boundary condition transforming unit transforming the biometric information, input to the biometric information input unit, into a boundary condition for performing computational fluid dynamics (CFD); a CFD performing unit performing cardiovascular CFD simulation of the target patient by applying the boundary condition transformed by the boundary condition transforming unit; and a result analyzing unit analyzing a cardiovascular disease risk of the target patient using a performing result from the CFD performing unit.

The biometric information input unit may receive biometric information including the target patient's gender, age, height, weight, systolic blood pressure, diastolic blood pressure, red blood cell density, blood calcium concentration, cardiovascular CT image, and upper airway CT image.

The boundary condition transforming unit may include: a first transforming unit calculating a pulsatile flow by using the systolic blood pressure and the diastolic blood pressure among the input biometric information and transforming the calculate pulsatile flow into an inlet boundary condition; a second transforming unit predicting an apnea-hypopnea index (AHI) of the target patient using the cardiovascular CT image and the upper airway CT image, among the input biometric information, and applying the predicted AHI value and the input biometric information to a previously stored conversion model formula to transform the same into an outlet boundary condition; and a third transforming unit calculating a blood viscosity using the red blood cell density and the blood calcium concentration among the input biometric information and transforming the calculated blood viscosity into a fluid viscosity condition.

The second transforming unit may perform three-dimensional (3D) modeling on an upper airway morphology using the cardiovascular CT image and the upper airway CT image, perform computational fluid analysis on the upper airway data, and analyze a performing result to calculate an AHI value of the target patient.

The second transforming unit may apply the following equation as a pre-stored transform model formula.

$$Q ( t ) = \frac{P ( t )}{R} + C \frac{dP ( t )}{dt}$$

The CFD performing unit may perform cardiovascular CFD simulation of the target patient by applying a Lattice Boltzmann method (LBM).

The result analyzing unit may receive the performing result from the CFD performing unit and analyze a cardiovascular disease risk of the target patient using a fractional flow reserve (FFR) and a wall shear stress (WSS) of the target patient.

In another general aspect, a cardiovascular disease risk analysis method considering sleep apnea factors, in which each operation is performed by a cardiovascular disease risk analysis system considering sleep apnea factors implemented by a computer to analyze a cardiovascular disease risk of a sleep apnea patient, includes: a biometric information input operation in which a biometric information input unit receives biometric information of a preset item for a target patient; a boundary condition transforming operation in which a boundary condition transforming unit transforms the biometric information input in the biometric information input operation into a boundary condition for performing computational fluid dynamics (CFD); a CFD performing operation in which a CFD performing unit performs cardiovascular CFD simulation of the target patient by applying the boundary condition transformed in the boundary condition transforming operation; and a risk analyzing operation in which a result analyzing unit analyzes a cardiovascular disease risk using a fractional flow reserve (FFR) and a wall shear stress (WSS) of the target patient, which are a performing result in the CFD performing operation, wherein, in the CFD performing operation, the cardiovascular CFD simulation is performed by applying Lattice Boltzmann method (LBM).

In the biometric information input operation, biometric information including the target patient's gender, age, height, weight, systolic blood pressure, diastolic blood pressure, red blood cell density, blood calcium concentration, cardiovascular CT image, and upper airway CT image may be received.

The boundary condition transforming operation may include: a first transforming operation of calculating a pulsatile flow by using the systolic blood pressure and the diastolic blood pressure among the input biometric information and transforming the calculated pulsatile flow into an inlet boundary condition; a second transforming operation of predicting an apnea-hypopnea index (AHI) of the target patient using the cardiovascular CT image and the upper airway CT image, among the input biometric information and applying the predicted AHI value and the input biometric information to a previously stored conversion model formula to transform the same into an outlet boundary condition; and

; and a third transforming operation of calculating a blood viscosity using the red blood cell density and the blood calcium concentration among the input biometric information and transforming the calculated blood viscosity into a fluid viscosity condition.

In the second transforming operation, three-dimensional (3D) modeling on an upper airway morphology may be performed using the cardiovascular CT image and the upper airway CT image, computational fluid analysis may be performed on the upper airway data, and a performing result may be analyzed to calculate an AHI value of the target patient.

Other features and aspects will be apparent from the following detailed description, the drawings, and the claims.

### BRIEF DESCRIPTION OF THE DRAWINGS

FIG. 1 is an exemplary configuration diagram illustrating a cardiovascular disease risk analysis system considering sleep apnea factors according to an exemplary embodiment of the present disclosure.

FIG. 2 is a view showing a basic conceptual diagram and formulas of computational fluid analysis applied to a cardiovascular disease risk analysis system considering sleep apnea factors according to an exemplary embodiment of the present disclosure.

FIG. 3 is an exemplary diagram illustrating a process of analyzing a cardiovascular disease risk of a target patient using the FFR, WSS, etc. of the target patient.

FIG. 4 is a flowchart illustrating a cardiovascular disease risk analysis method considering sleep apnea factors according to an exemplary embodiment of the present disclosure.

### DETAILED DESCRIPTION OF EXEMPLARY EMBODIMENTS

Hereinafter, a cardiovascular disease risk analysis system and method considering sleep apnea factors according to an exemplary embodiment of the present invention will be described in detail with reference to the accompanying drawings. The drawings are provided as examples in order to convey the spirit of the present invention to those skilled in the art. Therefore, the present invention is not limited to the drawings presented hereinafter and may be embodied in other forms. Throughout the specification, the same reference numbers will be used to refer to the same or like components.

If there are no other definitions in technical terms and scientific terms used here, the technical terms and scientific terms have the meanings commonly understood by those skilled in the art to which the present invention pertains, and in the following description and accompanying drawings, descriptions of known functions and components that may unnecessarily obscure the subject matter will be omitted.

In addition, the system refers to a set of components including devices, instruments, and means that are organized and regularly interact to perform necessary functions.

The cardiovascular disease risk analysis system and method considering sleep apnea factors according to an exemplary embodiment of the present disclosure are provided to analyze a cardiovascular disease risk of a sleep apnea patient through computational fluid dynamics (CFD), which is a field of fluid mechanics that analyzes fluid phenomena through numerical analysis and simulates them as in reality.

Such CFD is to simulate various fluid phenomena such as heat transfer, mass transfer, and chemical reaction through computer simulation, and the cardiovascular disease risk analysis system and method considering sleep apnea factors according to an exemplary embodiment of the present disclosure are optimized for small scale (μm˜cm) analysis, among the methods for implementing CFD, and thus, it is preferable to perform blood flow/vascular computer simulation using Lattice Boltzmann method (LBM) suitable for blood vessel modeling.

Since LBM constitutes a mesh in a grid format, the LBM may be easily applied to complex shapes such as blood vessels and advantageously increase a calculation speed through parallelization due to structural characteristics of the grid format.

The cardiovascular disease risk analysis system and method considering sleep apnea factors according to an exemplary embodiment of the present disclosure may proceed with blood flow modeling and derive flow factors such as FFR, WSS, etc. by performing CFD using the LBM, having advantages of assisting in the diagnosis of cardiovascular disease and being used in treatment planning.

In general, in order to perform computational fluid, a user should directly set all boundary conditions, so that a professional manpower is required.

However, in the cardiovascular disease risk analysis system and method considering sleep apnea factors according to an exemplary embodiment of the present disclosure, biometric information of a target patient (sleep apnea patient) may be received and automatically transformed into a boundary condition and a result may be derived through the performance of CFD through this, and thus, the result may be utilized even if a user is not a professional who is proficient in CFD.

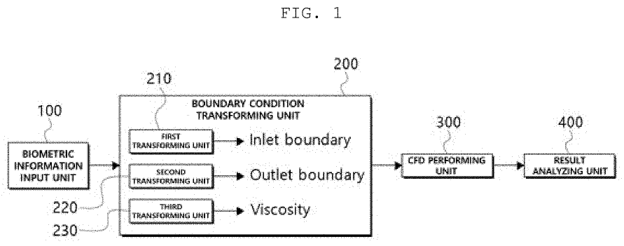

FIG. 1 is an exemplary configuration diagram illustrating a cardiovascular disease risk analysis system considering sleep apnea factors according to an exemplary embodiment of the present disclosure. A cardiovascular disease risk analysis system considering sleep apnea factors according to an exemplary embodiment of the present disclosure is described in detail.

As shown in FIG. 1, the cardiovascular disease risk analysis system considering sleep apnea factors according to an exemplary embodiment of the present disclosure may include a biometric information input unit 100, a boundary condition transforming unit 200, a CFD performing unit 300, and a result analyzing unit 400. Each component may be individually or integrally configured and operate in an arithmetic processing unit including a computer.

Each component is described in detail.

The biometric information input unit 100 preferably receives biometric information of a target patient (sleep apnea patient). The biometric information preferably includes the target patient's gender, age, height, weight, systolic blood pressure, diastolic blood pressure, red blood cell density (hematocrit), blood calcium concentration (calcification/calcium score), cardiovascular CT image, and upper airway CT image. It is most preferable that the biometric information input unit 100 receives the biometric information of the target patient based on the medical information that has already been collected/acquired, and in addition to the biometric information described above, various collectable biometric information may be utilized.

The boundary condition transforming unit 200 preferably transforms the boundary condition for performing CFD by using the biometric information of the target patient input to the biometric information input unit 100.

In detail, as shown in FIG. 1, the boundary condition transforming unit 200 includes a first transforming unit 210, a second transforming unit 220, and a third transforming unit 230.

The first transforming unit 210 is a component for transforming an inlet boundary condition, and it is preferred to set the inlet boundary condition by calculating a pulsatile flow using the systolic blood pressure and the diastolic blood pressure, among the biometric information input through the biometric information input unit 100.

The second transforming unit 220 is a component for transforming an outlet boundary condition, and it is preferred to set the outlet boundary condition by applying a pre-set transformation model formula using a windkessel model.

In detail, the second transforming unit 220 preferably predicts an AHI value of the target patient using the cardiovascular CT image and the upper airway CT image among the biometric information input through the biometric information input unit 100 and applies the predicted AHI value and the input biometric information to the transformation model formula.

A transformation model formula previously set using the windkessel model may be defined as Equation 1 below.

$$Q ( t ) = \frac{P ( t )}{R} + C \frac{dP ( t )}{dt} \qquad [ Equation 1 ]$$

Here, Q is blood flow rate, P is blood pressure, R, as a constant, is

$$\frac{dP}{Q} ,$$

f(calculated AHI value, input biometric information, . . . ), C, as a constant, is

$$\frac{dV}{dP} ,$$

f′(calculated AHI value, input biometric information, . . . ), dP is a blood pressure variance, and dV is a blood vessel volume variance.

As shown in the transformation model formula, the AHI value is required to calculate certain constants C and R, and the AHI value is a variable for diagnosing the severity of sleep apnea, which refers to the sleep apnea-hypopnea index.

Utilizing the AHI value of the sleep apnea patient for cardiovascular disease risk analysis, it was found that when sleep apnea worsens, breathing during sleep becomes rough and a blood pressure difference during sleep increases, and the blood vessel dilation/contraction is repeated due to the blood pressure difference to reduce vasodilation (distensibility). That is, a direct cause of the cardiovascular disease caused by sleep apnea is blood pressure variability due to sleep apnea. Day-night blood pressure variability due to changes in intrathoracic pressure during sleep puts a strain on blood vessels due to repeated expansion/contraction of blood vessels. This changes resistance and compliance by reducing vasodilation, as described above, thereby increasing the incidence of cardiovascular disease.

According to a survey, the incidence of cardiovascular disease in a general group with a low AHI value was 11%, whereas the incidence of a patient group with a high AHI value was 23%, which indicates that the incidence of cardiovascular disease due to sleep apnea is more than twice than that of the general group.

However, in the related art, in order to acquire an AHI value, sensing information acquired during actual sleep should be utilized/analyzed, but in the cardiovascular disease risk analysis system considering sleep apnea factors according to an exemplary embodiment of the present disclosure, the AHI value of the target patient may be predicted using the biometric information input through the biometric information input unit 100.

In detail, the second transforming unit 220 predicts an AHI value of the target patient using the cardiovascular CT image and the upper airway CT image, among the biometric information input through the biometric information input unit 100.

That is, preferably, three-dimensional (3D) modeling of the upper airway morphology is performed using the cardiovascular CT image and the upper airway CT image, and CFD is performed on upper airway shape data according to the performed 3D modeling and analyze a performing result to predict the AHI value of the target patient.

Here, velocity, pressure, pressure gradient, swirling strength, airway resistance, vorticity, helicity, wall shear stress, surface pressure, surface pressure gradient, deformation rate may be derived through the result of performing CFD on the upper airway shape data, which are then applied to a computational fluid or artificial intelligence algorithm once again through the CFD performing unit 300 to predict the AHI value of the target patient. This will be described later in detail.

In the computational fluid analysis technology for the upper airway shape data, modeling of the cardiovascular CT image and upper airway CT image in a 3D shape is first performed, and then the patient's respiration volume according to time is applied to an inlet and outlet of the model. (If it is difficult to directly measure the respiration volume of the patient, statistics may be used.) Thereafter, computational analysis is performed. Here, for the computational fluid analysis model used here, LBM may be used but it not limited thereto. Output factors are calculated based on computational analysis.

The third transforming unit 230, a component for transforming a fluid viscosity condition, sets the fluid viscosity condition by applying a transformation model formula previously set using the Carreau-Yasuda model.

In detail, the third transforming unit 230 calculates blood viscosity using the red blood cell density and blood calcium concentration, among the biometric information input through the biometric information input unit 100, and set as the blood viscosity as the fluid viscosity condition.

Here, the applied transformation model formula may be defined as Equation 2 below.

η*(w)=η_{∞}+(η₀−η_{∞})(1+(λw)^{a})^{(n-1)/a} [Equation 2]

Here, η₀ is viscosity at zero shear rate, η_{∞} is viscosity at infinite shear rate, λ is relaxation time, n is a power law index, and a is dimensionless parameter (2 in most cases).

Preferably, the CFD performing unit 300 predicts the AHI value of the target patient by performing CFD simulation by applying the boundary condition converted in the boundary condition transforming unit 200 using the LBM, that is, an inlet boundary condition, an outlet boundary condition, and the fluid viscosity condition.

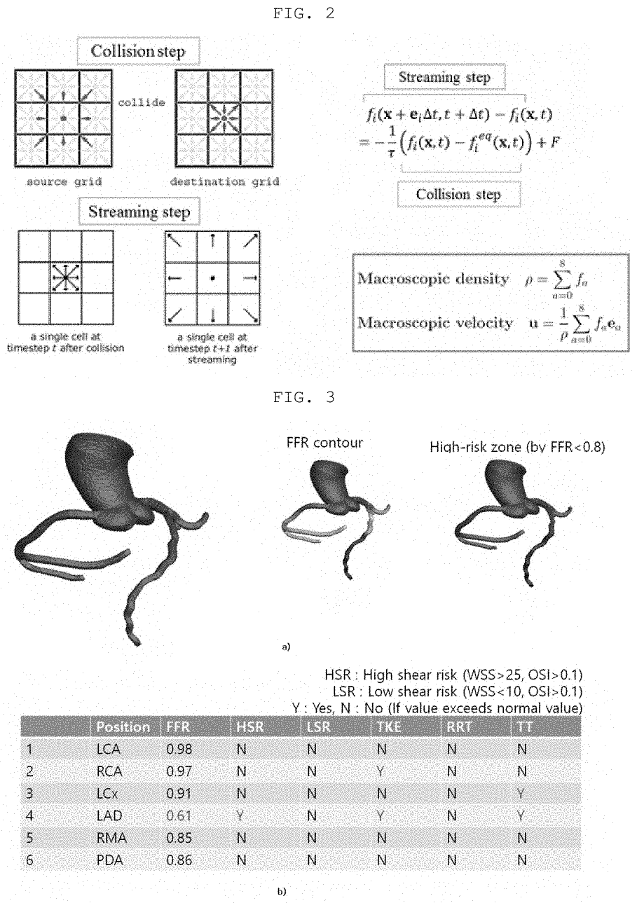

FIG. 2 is a view showing a basic conceptual diagram applied to the CFD performing unit 300 and equations of LBM, in which it is easy to use a complex shape model such as a blood vessel and it is easy to increase a calculation speed through parallelization due to a grid structure.

The AHI value is not a flow factor directly calculated through CFD and may be a respiration rate-related factor obtained through polysomnography. Based on a high correlation between CFD flow factors and AHI values, prediction of AHI values is performed based on CFD flow factors and the AHI value.

After performing learning on an algorithm with the flow factors acquired through the boundary condition transforming unit 200 as input and the AHI value measured directly from the patient as output, the boundary condition transforming unit 200 preferably predicts the AHI value of the target patent using a leaning model.

It is preferable to the cardiovascular disease risk of the target patient using the performing result of the CFD performing unit 300.

In detail, the result analyzing unit 400 receives the performing result from the CFD performing unit 300 and analyzes the cardiovascular disease risk of the target patient using the performing result information including a fractional flow reverse (FFR) and wall shear stress (WSS) of the target patient.

Here, FFR refers to a ratio of a proximal portion of the lesion, i.e., a mean aortic pressure, and a mean aortic pressure at a distal portion of the lesion, and WSS refers to a force acting on the vessel wall.

In addition to the FFR and WSS, as an example of the relationship between flow factors analyzed by CFD and stenosis, wall shear stress (WSS, Dynes/cm²) is an index for the vessel wall shear stress, that is, an influence that the vessel wall receives from friction with flow and is a traditional stenosis diagnostic factor. Excessively high or excessively low wall shear stress may cause a problem, and thus, boundary values >10 and <25 may be utilized.

In addition, an oscillatory shear index (OSI) is a factor indicating how a direction of the WSS changes according to the heartbeat, and as the corresponding value is higher, a direction of friction the blood vessel wall receives significantly changes to affect the development of stenosis. A boundary value thereof is >0.1.

Area with OSI <0.1(%) is an index indicating how widely a region with high OSI is distributed throughout the blood vessel, and a boundary value thereof is >3.

Turbulent kinetic energy (TKE, mJ) is a numerical value expressing information on turbulence of blood flow, and as the numeral value is higher, TKE flows more chaotically, increasing possibility of stenosis development. A boundary value thereof is >7.

Relative residence time (RRT, 1/Pa) is an index expressing how long the blood stays in one place, and as the corresponding value is higher, it is determined that the flow is biologically stopped and that a possibility of revealing the stenosis development mechanism is considered to be large. A boundary value thereof is >4.

The result analyzing unit 400 preferably analyzes the cardiovascular disease risk of the target patient using the boundary value set in advance for each result variable, based on the performing result of the CFD performing unit 300.

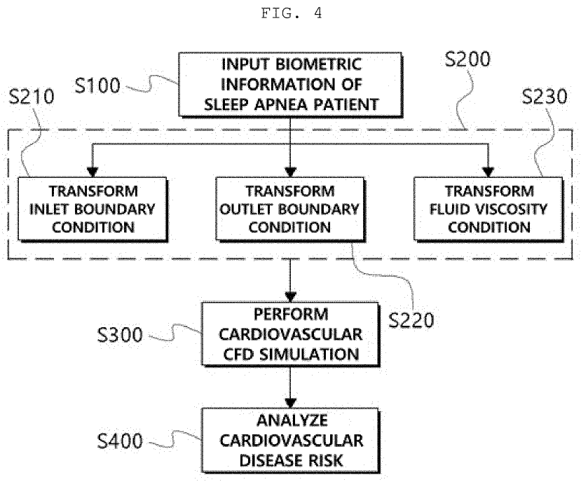

FIG. 3 shows a process of analyzing a cardiovascular disease risk of a target patient using the FFR, WSS, etc. of the target patient upon receiving the performing result from the CFD performing unit 300 through the result analyzing unit 400.

FIG. 4 is a flowchart illustrating a cardiovascular disease risk analysis method considering sleep apnea factors according to an exemplary embodiment of the present disclosure. The cardiovascular disease risk analysis method is described in detail.

As shown in FIG. 4, the cardiovascular disease risk analysis method considering sleep apnea factors according to an exemplary embodiment of the present disclosure includes a biometric information input operation (S100), a boundary condition transforming operation (S200), a CFD performing operation (S300), and a risk analyzing operation (S400).

Each operation is described in detail.

In the biometric information input operation (S100), the biometric information input unit 100 receives biometric information of a preset item for a target patient (sleep apnea patient), and the biometric information may include the target patient's gender, age, and height, weight, systolic blood pressure, diastolic blood pressure, red blood cell density (Hematocrit), blood calcium concentration (calcification/calcium score), cardiovascular CT image, and upper airway CT image.

It is most preferable to receive (input) the biometric information of the target patient based on already collected/acquired medical information, and in addition to the biometric information described above, various collectable biometric information may be utilized.

In the boundary condition transforming operation (S200), the boundary condition transforming unit 200 transforms into a boundary condition for performing CFD using the biometric information input in the biometric information input operation (S100).

In detail, the boundary condition transforming operation (S200) may include a first transforming operation (S210), a second transforming operation (S220) and a third transforming operation (S230), as shown in FIG. 4.

The first transforming operation (S210) is an operation of transforming an inlet boundary condition, in which a pulsatile flow is calculated and set as the inlet boundary condition using the systolic blood pressure and the diastolic blood pressure, among the biometric information input in the biometric information input operation (S100).

The second transforming operation (S220) is an operation of transforming an outlet boundary condition, in which the outlet boundary condition is set by applying a pre-set transformation model formula using a windkessel model.

In detail, in the second transforming operation (S220), the AHI value of the target patient is predicted using the cardiovascular CT image and the upper airway CT image, among the biometric information input in the biometric information input operation (S100), and the predicted AHI value and the input biometric information are applied to the transformation model formula.

The transformation model formula previously set using the windkessel model may be defined by Equation 1 above, and as shown in the transformation model formula, the AHI value is required to calculate certain constants C and R, and the AHI value is a variable for diagnosing the severity of sleep apnea and refers to the apnea-hypopnea index during sleep.

Utilizing the AHI value of the sleep apnea patient for cardiovascular disease risk analysis, it was found that when sleep apnea worsens, breathing during sleep becomes rough and a blood pressure difference during sleep increases, and the blood vessel dilation/contraction is repeated due to the blood pressure difference to reduce vasodilation (distensibility). That is, a direct cause of the cardiovascular disease caused by sleep apnea is blood pressure variability due to sleep apnea. Day-night blood pressure variability due to changes in intrathoracic pressure during sleep puts a strain on blood vessels due to repeated expansion/contraction of blood vessels. This changes resistance and compliance by reducing vasodilation, as described above, thereby increasing the incidence of cardiovascular disease.

According to a survey, the incidence of cardiovascular disease in a general group with a low AHI value was 11%, whereas the incidence of a patient group with a high AHI value was 23%, which indicates that the incidence of cardiovascular disease due to sleep apnea is more than twice than that of the general group.

However, in the related art, in order to acquire an AHI value, sensing information acquired during actual sleep should be utilized/analyzed, but in the cardiovascular disease risk analysis system considering sleep apnea factors according to an exemplary embodiment of the present disclosure, the AHI value of the target patient may be predicted using the biometric information input through the biometric information input unit 100.

To this end, in the second transforming operation (S220), the AHI value of the target patient is predicted using the cardiovascular CT image and the upper airway CT image, among the biometric information input in the biometric information input operation (S100).

Preferably, three-dimensional (3D) modeling of the upper airway morphology is performed using the cardiovascular CT image and the upper airway CT image, and CFD is performed on upper airway shape data according to the performed 3D modeling and analyze a performing result to predict the AHI value of the target patient.

Here, velocity, pressure, pressure gradient, swirling strength, airway resistance, vorticity, helicity, wall shear stress, surface pressure, surface pressure gradient, deformation rate may be derived through the result of performing CFD on the upper airway shape data, which are then applied to a computational fluid or artificial intelligence algorithm once again to predict the AHI value of the target patient.

In other words, in the computational fluid analysis technology for the upper airway shape data, modeling of the cardiovascular CT image and upper airway CT image in a 3D shape is first performed, and then the patient's respiration volume according to time is applied to an inlet and outlet of the model. (If it is difficult to directly measure the respiration volume of the patient, statistics may be used.) Thereafter, computational analysis is performed. Here, for the computational fluid analysis model used here, LBM may be used but it is not limited thereto. Output factors are calculated based on computational analysis.

The third transforming operation (S230) is an operation of transforming a fluid viscosity condition, and the fluid viscosity condition is set by applying a pre-set transformation model formula using the Carreau-Yasuda model.

In detail, in the third transforming operation (S230), blood viscosity is calculated using the red blood cell density and blood calcium concentration, among the biometric information input through the biometric information input unit 100, and set as the fluid viscosity condition.

Here, the applied transformation model formula may be defined as Equation 2 above.

In the CFD performing operation (S300), the CFD performing unit 300 performs a cardiovascular CFD simulation of the target patient by applying the boundary conditions transformed in the boundary condition transforming operation (S200).

In other words, in the CFD performing operation (S300), the CFD performing unit 300 may perform the CFD simulation by applying the boundary conditions (inlet boundary condition, outlet boundary condition, and fluid viscosity condition) transformed in the boundary condition transforming operation (S200) using the LBM.

FIG. 2 is a view showing a basic conceptual diagram applied to the CFD performing unit 300 and equations of LBM, in which it is easy to use a complex shape model such as a blood vessel and it is easy to increase a calculation speed through parallelization due to a grid structure.

The AHI value is not a flow factor directly calculated through CFD and may be a respiration rate-related factor obtained through polysomnography. Based on a high correlation between CFD flow factors and AHI values, prediction of AHI values is performed based on CFD flow factors and the AHI value.

After performing learning on an algorithm with the flow factors acquired through the boundary condition transforming unit 200 as input and the AHI value measured directly from the patient as output, the boundary condition transforming unit 200 preferably predicts the AHI value of the target patent using a leaning model.

In the risk analyzing operation (S400), the result analyzing unit 400 analyzes the cardiovascular disease risk using the performing result information including a fractional flow reverse (FFR) of the target patient and a wall shear stress (WSS) of the target patient, which is a performing result of the CFD performing operation (S300).

Here, FFR refers to a ratio of a proximal portion of the lesion, i.e., a mean aortic pressure, and a mean aortic pressure at a distal portion of the lesion, and WSS refers to a force acting on the vessel wall.

In addition to the FFR and WSS, as an example of the relationship between flow factors analyzed by CFD and stenosis, wall shear stress (WSS, Dynes/cm²) is an index for the vessel wall shear stress, that is, an influence that the vessel wall receives from friction with flow and is a traditional stenosis diagnostic factor. Excessively high or excessively low wall shear stress may cause a problem, and thus, boundary values >10 and <25 may be utilized.

In addition, an oscillatory shear index (OSI) is a factor indicating how a direction of the WSS changes according to the heartbeat, and as the corresponding value is higher, a direction of friction the blood vessel wall receives significantly changes to affect the development of stenosis. A boundary value thereof is >0.1.

Area with OSI <0.1(%) is an index indicating how widely a region with high OSI is distributed throughout the blood vessel, and a boundary value thereof is >3.

Turbulent kinetic energy (TKE, mJ) is a numerical value expressing information on turbulence of blood flow, and as the numeral value is higher, TKE flows more chaotically, increasing possibility of stenosis development. A boundary value thereof is >7.

Relative residence time (RRT, 1/Pa) is an index expressing how long the blood stays in one place, and as the corresponding value is higher, it is determined that the flow is biologically stopped and that a possibility of revealing the stenosis development mechanism is considered to be large. A boundary value thereof is >4.

The result analyzing unit 400 preferably analyzes the cardiovascular disease risk of the target patient using the boundary value set in advance for each result variable, based on the performing result of the CFD performing unit 300.

In other words, in the cardiovascular disease risk analysis system and method considering sleep apnea factors according to an exemplary embodiment of the present disclosure, biometric information of a target patient (sleep apnea patient) may be received and automatically transformed into a boundary condition and a result may be derived through the performance of CFD through this, and thus, overall blood vessel pre-screening and treatment planning may be made by utilizing the result even if a user is not a professional manpower who is proficient in CFD.

The cardiovascular disease risk analysis system and method considering sleep apnea factors of the present disclosure according to the configuration as described above may calculate a patient's sleep apnea severity diagnostic variable value using the patient's biometric information which is easy to collect, even without a complicated measurement process, and perform computational fluid analysis simulation through setting of a boundary condition for blood vessel/blood flow modeling by using the patient's calculated sleep apnea severity diagnostic variable value, thereby analyzing a cardiovascular disease risk.

In particular, it is possible to pre-screen the overall cardiovascular system based on the computational fluid analysis, which may be used as decision-making data for patient's treatment planning.

As described above, in the present disclosure, specific matters such as specific components and the like have been described with reference to the limited exemplary embodiment drawings, but this is only provided to help a more general understanding of the present disclosure, and the present disclosure is not limited to the above exemplary embodiment, and various modifications and variations may be from these descriptions by those skilled in the art to which the present disclosure pertains.

Therefore, the spirit of the present disclosure should not be limited to the exemplary embodiments described above, and not only the claims to be described later, but also all those with equivalent or equivalent modifications to the claims are within the scope of the spirit of the present disclosure.

### DETAILED DESCRIPTION OF MAIN ELEMENTS

100: bio-information input unit
200: boundary condition transforming unit
210: first transforming unit 220: second transforming unit
230: third transforming unit
300: CFD performing unit
400: result analyzing unit

What is claimed is:

1. A system for analyzing a cardiovascular disease risk of a sleep apnea patient, the system comprising:
a biometric information input unit receiving biometric information of a target patient;
a boundary condition transforming unit transforming the biometric information, input to the biometric information input unit, into a boundary condition for performing computational fluid dynamics (CFD);
a CFD performing unit performing cardiovascular CFD simulation of the target patient by applying the boundary condition transformed by the boundary condition transforming unit; and
a result analyzing unit analyzing a cardiovascular disease risk of the target patient using a performing result from the CFD performing unit.

2. The system of claim 1, wherein the biometric information input unit receives biometric information including the target patient's gender, age, height, weight, systolic blood pressure, diastolic blood pressure, red blood cell density, blood calcium concentration, cardiovascular CT image, and upper airway CT image.

3. The system of claim 2, wherein
the boundary condition transforming unit includes:
a first transforming unit calculating a pulsatile flow by using the systolic blood pressure and the diastolic blood pressure among the input biometric information and transforming the calculated pulsatile flow into an inlet boundary condition;
a second transforming unit predicting an apnea-hypopnea index (AHI) of the target patient using the cardiovascular CT image and the upper airway CT image, among the input biometric information, and applying the predicted AHI value and the input biometric information to a previously stored conversion model formula to transform the same into an outlet boundary condition;
a third transforming unit calculating a blood viscosity using the red blood cell density and the blood calcium concentration among the input biometric information and transforming the calculated blood viscosity into a fluid viscosity condition.

4. The system of claim 3, wherein the second transforming unit performs three-dimensional (3D) modeling on an upper airway morphology using the cardiovascular CT image and the upper airway CT image, performs computational fluid analysis on the upper airway data, and analyzes a performing result to calculate an AHI value of the target patient.

5. The system of claim 4, wherein
the second transforming unit applies the following equation as a pre-stored transform model formula.
$$Q ( t ) = \frac{P ( t )}{R} + C \frac{dP ( t )}{dt}$$
wherein
Q is blood flow rate,
P is blood pressure,
R, as a constant, is
$$\frac{dP}{Q} ,$$
f(calculated AHI value, input biometric information, . . . ),
C, as a constant, is
$$\frac{dV}{dP} ,$$
f′(calculated AHI value, input biometric information, . . . ),
dP is a blood pressure variance, and
dV is a blood vessel volume variance.

6. The system of claim 1, wherein the CFD performing unit performs cardiovascular CFD simulation of the target patient by applying a Lattice Boltzmann method (LBM).

7. The system of claim 1, wherein the result analyzing unit receives the performing result from the CFD performing unit and analyzes a cardiovascular disease risk of the target patient using a fractional flow reserve (FFR) and a wall shear stress (WSS) of the target patient.

8. A cardiovascular disease risk analysis method considering sleep apnea factors, in which each operation is performed by a cardiovascular disease risk analysis system considering sleep apnea factors implemented by a computer to analyze a cardiovascular disease risk of a sleep apnea patient, the method comprising:
a biometric information input operation in which a biometric information input unit receives biometric information of a preset item for a target patient;
a boundary condition transforming operation in which a boundary condition transforming unit transforms the biometric information input in the biometric information input operation into a boundary condition for performing computational fluid dynamics (CFD);
a CFD performing operation in which a CFD performing unit performs cardiovascular CFD simulation of the target patient by applying the boundary condition transformed in the boundary condition transforming operation; and
a risk analyzing operation in which a result analyzing unit analyzes a cardiovascular disease risk using a fractional flow reserve (FFR) and a wall shear stress (WSS) of the target patient, which are a performing result in the CFD performing operation,
wherein, in the CFD performing operation, the cardiovascular CFD simulation is performed by applying Lattice Boltzmann method (LBM).

9. The method of claim 8, wherein, in the biometric information input operation, biometric information including the target patient's gender, age, height, weight, systolic blood pressure, diastolic blood pressure, red blood cell density, blood calcium concentration, cardiovascular CT image, and upper airway CT image is received.

10. The method of claim 9, wherein
the boundary condition transforming operation includes:
a first transforming operation of calculating a pulsatile flow by using the systolic blood pressure and the diastolic blood pressure among the input biometric information and transforming the calculated pulsatile flow into an inlet boundary condition;
a second transforming operation of predicting an apnea-hypopnea index (AHI) of the target patient using the cardiovascular CT image and the upper airway CT image, among the input biometric information and applying the predicted AHI value and the input biometric information to a previously stored conversion model formula to transform the same into an outlet boundary condition; and
a third transforming operation of calculating a blood viscosity using the red blood cell density and the blood calcium concentration among the input biometric information and transforming the calculated blood viscosity into a fluid viscosity condition.

11. The method of claim 10, wherein, in the second transforming operation, three-dimensional (3D) modeling on an upper airway morphology is performed using the cardiovascular CT image and the upper airway CT image, computational fluid analysis is performed on the upper airway data, and a performing result is analyzed to calculate an AHI value of the target patient.
